(19) United States
(12) Patent Application Publication
(10) Pub. No.: US 2024/0201195 A1
(43) Pub. Date: Jun. 20, 2024

(54) Process for Detecting Nucleus-Containing Cells in a Sample Liquid of a Patient using a Microfluidic Device and Microfluidic Device

(71) Applicant: Robert Bosch GmbH, Stuttgart (DE)

(72) Inventors: Samir Kadic, Langenau (DE); Tianxing Du, Freising (DE); Franz Laermer, Weil Der Stadt (DE); Jochen Hoffmann, Renningen (DE); Anne Serout, Aidlingen (DE)

(21) Appl. No.: 18/555,486

(22) PCT Filed: Apr. 20, 2022
(86) PCT No.: PCT/EP2022/060378

§ 371 Date: Oct. 13, 2023

(30) Foreign Application Priority Data
Apr. 20, 2021 (DE) 10 2021 203 897.2

(51) Int. Cl. G01N 33/574; B01L 3/00; G01N 33/569

(52) U.S. Cl. CPC G01N 33/57496; B01L 3/502715; B01L 3/502761; G01N 33/56966; B01L 2200/025; B01L 2200/0652; B01L 2200/10; B01L 2200/16; B01L 2300/0819; B01L 2400/0487

(57) ABSTRACT

A process for detecting nucleus-containing cells in a sample liquid of a patient using a microfluidic device is disclosed. The process includes (i) providing a mixing signal to a mixing device, wherein the mixing signal causes mixing of the sample liquid with a lysis buffer in a mixing chamber of the microfluidic device in order to obtain a lysate, (ii) outputting an application signal which causes application of the lysate onto a carrier substrate of the microfluidic device in order to obtain a cell sediment and cell suspension of the lysate, and (iii) identifying the nucleus-containing cells from the cell sediment.

### PRIOR ART

The invention proceeds from a process for detecting nucleus-containing cells in a sample liquid of a patient and a microfluidic device belonging to the class of patent specified in the independent claims. The subject matter of the present invention is also a computer program.

Circulating Tumor Cells (CTCs) have established themselves over the past few years as promising and clinically relevant biomarkers for the study of malignant tumors. Their detection as early as possible in suitable human body fluids, usually blood, but also lymph fluid, for example, can be analyzed, and offers numerous advantages over typically invasive tissue biopsies. It is therefore one of the research focuses of modern oncology, known as Liquid Biopsy. For example, a time-resolved quantification of CTCs per normalized volume of a cancer patient's blood allows precise, real-time monitoring of the patient's disease progression, the ability to make treatment decisions adapted to the individual disease situation, and even to provide prognoses of progression-free survival.

WO 2012/138882 A2 describes a microfluidic device with microcavities for detecting biological cells using magnetic elements.

### DISCLOSURE OF THE INVENTION

In light of this, with the approach presented here, an improved process, furthermorean improved microfluidic device using this process, and finally a corresponding computer program according to the main claims are presented. By the measures listed in the dependent claims, advantageous developments and improvements of the apparatus specified in the independent claim are possible.

By the approach presented here, a medical analysis of a patient sample can advantageously be performed automatically, which can advantageously be associated with time-critical examinations. Accordingly, the approach presented can save time.

A process for detecting nucleus-containing cells in a sample liquid of a patient using a microfluidic device is presented, wherein the process comprises a step of providing, a step of outputting, and a step of identifying. The step of providing involves providing a mixing signal to an interface with a mixing device, wherein the mixing signal causes mixing of the sample liquid with a lysis buffer in a mixing chamber of the microfluidic device in order to obtain a lysate. The step of outputting involves outputting an application signal which causes application of the lysate onto a carrier substrate of the microfluidic device to obtain a cell sediment and a cell suspension of the lysate. The step of identifying involves identifying the nucleus-containing cells from the cell sediment.

The process can advantageously be performed automatically in order to detect the nucleus-containing cells, such as tumor cells, leukocytes, endothelial cells, or stem cells. According to the approach presented here, “nucleus-containing cells” can be understood to mean cells that have a nucleus. Specifically, these cells contain genetic information in the nucleus that allows the cell to be reproduced from the genetic information in this nucleus. Cells without a nucleus, on the other hand, can be, for example, red blood cells (erythrocytes) or platelets (thrombocytes). For example, the sample liquid can be a blood sample of the patient. For example, the microfluidic device can be formed as, or at least comprise, a lab-on-chip cartridge. For example, the sample liquid can be pumped back and forth in the mixing chamber in or before the step of providing so that a uniform concentration and distribution of the lysate occurs. The lysis buffer may, for example, be formed as a chemical fluid that has such properties in order to be able to separate erythrocytes, for example, from leukocytes, and additionally or alternatively from tumor cells. Advantageously, the lysis buffer can be formed as an ammonium chloride lysis buffer (ACK lysis buffer), for example to produce a difference in density of the cells. Advantageously, the lysate can be moved within the mixing chamber for a predetermined period of time, in particular at least five minutes, to achieve a homogeneous concentration of the constituents of the lysate. For example, the carrier substrate can be formed as a chip. Advantageously, the lysate is applied onto the carrier substrate where it can rest for a predetermined period of time. During this period of time, sedimentation can advantageously occur so that the cell sediment, which means still intact cells, can deposit on the carrier substrate and ultimately be identified in the step of identifying.

According to one embodiment, the process can comprise a step of washing the lysate prior to the step of identifying using a wash buffer to cause optical transparency of the lysate. Advantageously, the lysate can thereby be purified such that it becomes clearer and, correspondingly, a more accurate identification of the nucleus-containing cells can be carried out. As a result, sources of failure can thereby be reduced. The wash buffer used for this purpose can advantageously be formed as a phosphate buffered saline (PBS).

Furthermore, in the step of washing, the lysate can be washed using the wash buffer which is isotonic and additionally or alternatively pH neutral. Advantageously, this can ensure and guarantee that the nucleus-containing cells to be identified are not damaged.

According to one embodiment, in the step of providing, the mixing signal can be provided which causes mixing of an amount of the lysis buffer that is dependent on an amount of the sample liquid. Advantageously, a corresponding mixing ratio of the sample liquid and the lysis buffer can be predetermined. Furthermore, the amounts of the two liquids can advantageously be provided automatically.

Furthermore, in the step of identifying, the nucleus-containing cells from the cell sediment can be optically detected and additionally or alternatively quantified. Advantageously, a disease, for example, and additionally or alternatively a severity of the disease can be determined as a result.

According to one embodiment, in the step of providing, the mixing signal can be provided to mix the sample liquid with the lysis buffer. In so doing, the lysis buffer can comprise a fluorescent dye for determining a type of cell of the nucleus-containing cells. Advantageously, the dye can act on the nucleus-containing cells of the lysate, such that the cell sediment differs from the cell suspension in color, and thus it is advantageously easier to detect whether and in what amount, for example, tumor cells are present in the lysate.

According to one embodiment, the process can comprise a step of supplying the fluorescent dye into the lysis buffer prior to the step of providing. Advantageously, this can result in time savings.

The process can also comprise a step of introducing the sample liquid into the microfluidic device. Advantageously, the introduction of the sample liquid can be performed manually by a user or alternatively by a machine.

It is further advantageous to have an embodiment of the approach presented here as a process for detecting nucleus-containing cells in a sample liquid of a patient using a variant of a microfluidic device presented here, wherein the process comprises a step of mixing the sample liquid with a lysis buffer in a mixing chamber of the microfluidic device in order to obtain a lysate. Further, the process comprises a step of applying the lysate to a carrier substrate of the microfluidic device to obtain a cell sediment and a cell suspension of the lysate. Finally, the process comprises a step of identifying the nucleus-containing cells from the cell sediment. Such an embodiment, also allows the advantages of the approach presented here to be realized quickly and efficiently.

In this case, it is advantageous if the process comprises a step of washing the lysate prior to the step of identifying using a wash buffer in order to cause an optical transparency of the lysate.

In this case, the wash buffer is, for example, isotonic and/or pH-neutral. The aforementioned advantages result from this.

In addition, it is advantageous if the step of mixing involves mixing an amount of the lysis buffer that is dependent on an amount of the sample liquid.

The aforementioned advantages result from this.

Furthermore, it is advantageous for the nucleus-containing cells from the cell sediment to be optically detected and/or quantified in the step of identifying. The aforementioned advantages result from this.

It is also advantageous if, in the step of mixing the sample liquid with the lysis buffer, the lysis buffer comprises a fluorescent dye for determining a cell type of the nucleus-containing cells, in particular wherein a step of supplying the fluorescent dye into the lysis buffer is provided prior to the step of mixing. The aforementioned advantages result from this.

It is further advantageous if the process comprises a step of introducing the sample liquid into the microfluidic device. The aforementioned advantages result from this.

Furthermore, a microfluidic device for detecting nucleus-containing cells in a sample liquid of a patient is presented, wherein in particular the microfluidic device can be formed as a lab-on-chip cartridge. The microfluidic device comprises a mixing chamber for receiving the sample liquid and a lysis buffer in order to obtain a lysate. Furthermore, the microfluidic device comprises a carrier substrate for receiving the lysate to obtain a cell sediment and a cell suspension of the lysate as well as a detection chamber. The carrier substrate is arranged and/or can be arranged in the detection chamber.

Advantageously, the microfluidic device can be used in conjunction with rapid tests, as it enables, with a time advantage, a procedural flow of a process for detecting nucleus-containing cells in a patient sample in any of the aforementioned variants. Advantageously, the microfluidic device can be configured to, for example, analyze a blood sample. For example, the carrier substrate can be chip-like.

According to one embodiment, the detection chamber can have a height that is less than a width and a length of the detection chamber. For example, the detection chamber can have a height of 320 microns and, for example, a square base area with, for example, edge lengths of 12.5 mm×12.5 mm. Additionally or optionally, dimensions of the mixing chamber can be 13 mm×13 mm×10 mm.

According to one embodiment, the carrier substrate can comprise a plurality of microcavities. Advantageously, the cell sediment can deposit in the individual microcavities. Furthermore, the individual microcavities can be arranged in a honeycomb shape.

Furthermore, the microfluidic device can comprise a buffer storage chamber configured to store the lysis buffer and output it into the mixing chamber. The buffer storage chamber can advantageously have a predetermined capacity, which can correspond to the required amount of lysis buffer.

An evaluation device for evaluating a cell sediment in a microfluidic device in a previously mentioned variant is further presented, wherein the evaluation device comprises a mixing device and an identification unit. The mixing device is configured to mix the sample liquid with the lysis buffer in the mixing chamber in order to obtain a lysate. The identification unit is configured to identify the nucleus-containing cells from the cell sediment.

Advantageously, the evaluation device can comprise a control unit or a control device for controlling and/or performing the steps of the process in any of the aforementioned variants. The mixing device can comprise a pumping unit or be formed as such. Advantageously, this allows the sample liquid and the lysis buffer to be mixed to form a homogeneous lysate. The identification unit can advantageously comprise or be formed as a microscope unit and at least one light source.

The aforementioned process can be implemented, for example, in software or hardware or in a mixed form of software and hardware, for example in a control device or a control unit.

The approach presented here further creates a control unit configured to carry out, control or implement the steps of a variant of a process presented here in corresponding devices. This design variant of the invention in the form of a control unit can likewise achieve the underlying object of the invention quickly and efficiently.

For this purpose, the control unit can comprise at least one computing unit for processing signals or data, at least one memory unit for storing signals or data, at least one interface with a sensor or an actuator for reading in sensor signals from the sensor or for outputting control signals to the actuator, and/or at least one communication interface for reading in or outputting data embedded in a communication protocol. The computing unit can be a signal processor, a microcontroller or the like, for example, wherein the memory unit can be a flash memory, an EEPROM or a magnetic memory unit. The communication interface can be configured to read in or output data wirelessly and/or by wire, wherein a communication interface capable of reading in or outputting data transmitted by wire can read said data, for example electrically or optically, from a corresponding data transmission line or output the data to a corresponding data transmission line.

A control unit can be understood here to be an electrical device that processes sensor signals and outputs control signals and/or data signals as a function thereof. The control unit can have an interface, which can be formed by hardware and/or software. In a hardware design, the interfaces can, for example, be part of a so-called system ASIC, which contains various functions of the control unit. However, it is also possible that the interfaces are separate, integrated circuits or at least partially consist of discrete structural elements. Given a software design, the interfaces can be software modules provided on, e.g., a microcontroller in addition to other software modules.

A computer program product or a computer program with program code that can be stored on a machine-readable carrier or storage medium, such as a semiconductor memory, a hard disk memory, or an optical memory, and that is used to carry out, implement, and/or control the steps of the process according to one of the embodiments described above is advantageous as well, in particular when the program product or program is executed on a computer or an apparatus.

Embodiment examples of the approach presented here are shown in the drawings and explained in greater detail in the following description. The drawings show:

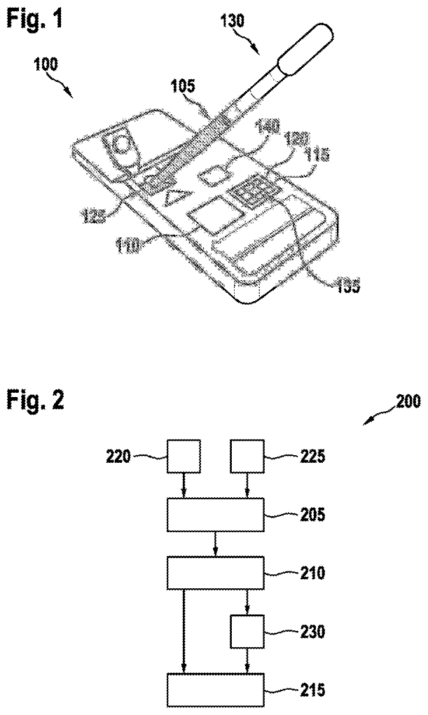

FIG. 1 a schematic view of a microfluidic device according to an embodiment example of the invention;

FIG. 2 a flowchart of a process according to an embodiment example for detecting nucleus-containing cells in a sample liquid of a patient;

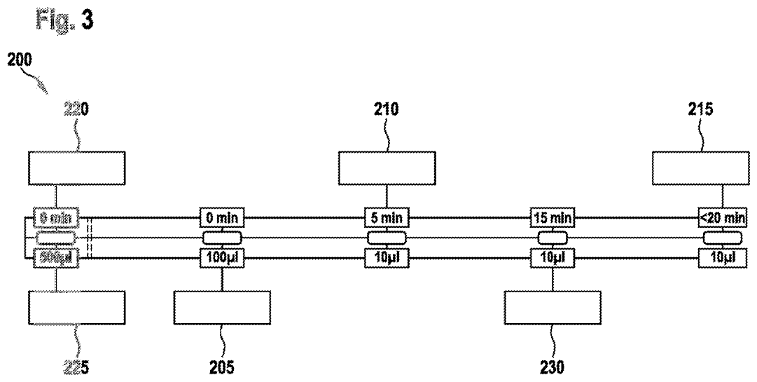

FIG. 3 a flowchart of a process according to an embodiment example for detecting nucleus-containing cells in a sample liquid of a patient;

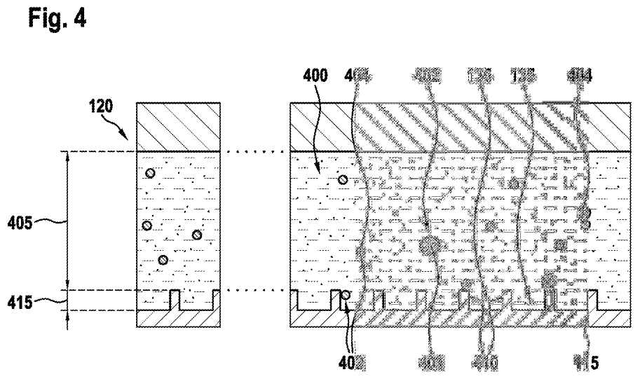

FIG. 4 a schematic embodiment example of a cross-section of a detection chamber of a microfluidic device;

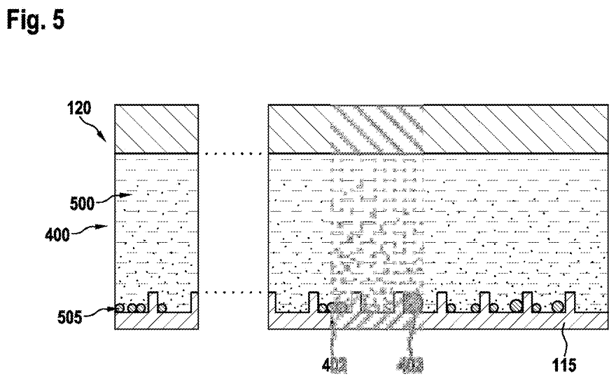

FIG. 5 a schematic embodiment example of a cross-section of a detection chamber of a microfluidic device;

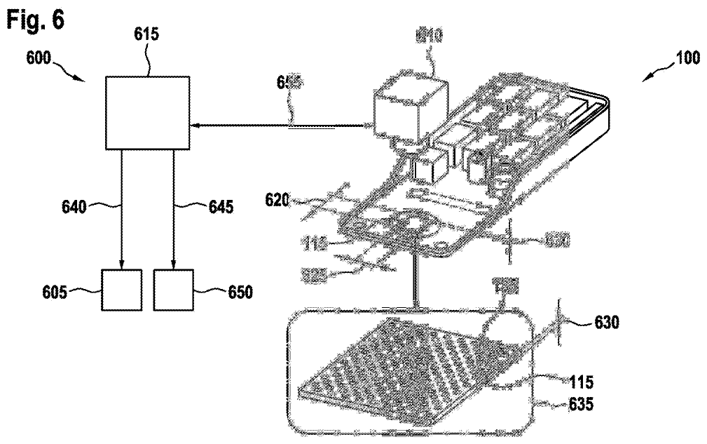

FIG. 6 an evaluation device according to an embodiment example and a microfluidic device;

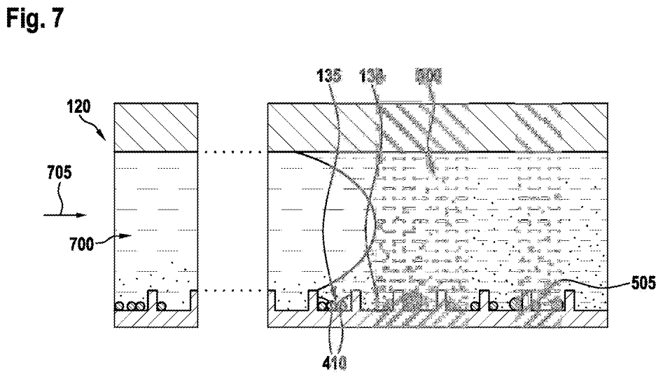

FIG. 7 a schematic embodiment example of a cross-section of a detection chamber of a microfluidic device;

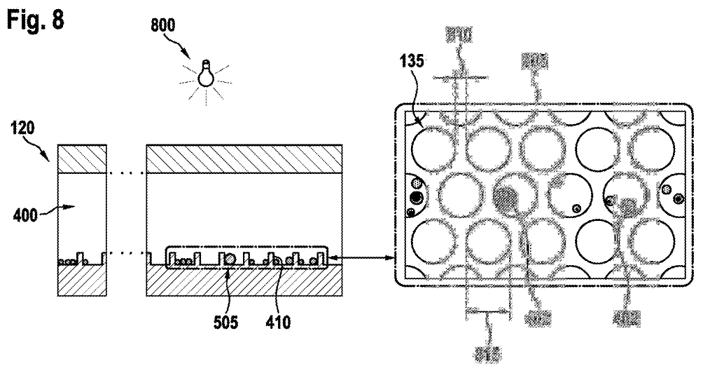

FIG. 8 a schematic embodiment example of a cross-section of a detection chamber of a microfluidic device; and

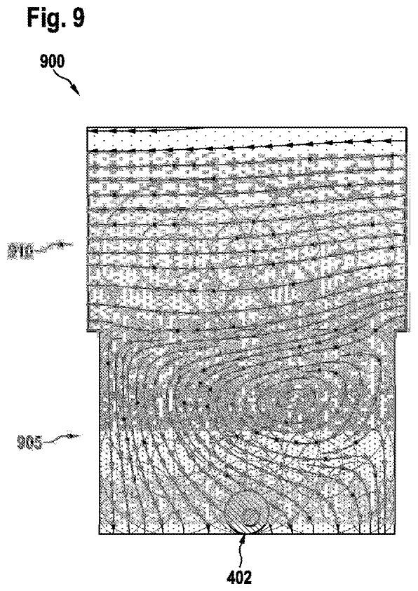

FIG. 9 a schematic illustration of a flow path in a microcavity according to an embodiment example.

In the following description of favorable embodiment examples of the present invention, identical or similar reference numbers are used for the elements shown in the various figures and acting similarly, wherein a repeated description of these elements is dispensed with.

FIG. 1 shows a schematic illustration of a microfluidic device 100 according to an embodiment example of the invention. The microfluidic device 100 is configured to detect nucleus-containing cells in a sample liquid 105 of a patient. In particular, the microfluidic device 100 is formed as a lab-on-chip cartridge. The microfluidic device 100 comprises a mixing chamber 110 for receiving the sample liquid 105 and a lysis buffer, which together form a lysate. In this regard, it is generally noted that the elements shown in FIG. 1 substantially reflect only an overview of components that can be realized on the microfluidic device 100; a more detailed representation or description of the location or position of these elements or components cannot be seen in FIG. 1. For this purpose, the functional design including the position of the individual components in relation to each other is described in more detail below. Furthermore, the microfluidic device 100 comprises a carrier substrate 115 for receiving the lysate in order to obtain a cell sediment and a cell suspension of the lysate as well as a detection chamber 120. The carrier substrate 115 is arranged and/or can be arranged in the detection chamber 120. The microfluidic device 100 is configured to, for example, shorten an analysis duration of the sample liquid 105, as it can be used as a cartridge in conjunction with, for example, rapid tests. For example, the sample liquid 105 is realized as the patient's blood, which is tested for tumor cells (CTCs), for example. Analysis of the sample liquid 105 is performed automatically, for example, so that the analysis duration is also reduced, which is particularly advantageous in time-critical situations. According to this embodiment example, the microfluidic device 100 comprises an inlet interface 125 configured to admit the sample liquid 105 into an interior of the device 100, where it is ultimately examined by a process for detecting nucleus-containing cells, as further explained in one of the following figures. For example, the sample liquid 105 is manually or alternatively automatically admitted into the microfluidic device 100 using a filling device 130, for example, by means of a pipette.

According to this embodiment example, the mixing chamber 110 is shallower compared to the detection chamber 120 according to this embodiment example. According to an alternative embodiment, it is conceivable that the dimensions of the chambers 110, 120 differ from one another. In this regard, the carrier substrate 115 is arranged in the detection chamber 120, which is realized or can be realized, for example, as a removable or insertable microchip. Alternatively, the carrier substrate 115 is formed as a fixed chip, for example. The carrier substrate 115 only optionally comprises a plurality of microcavities 135, which are formed as recesses on an area of the carrier substrate 115, for example. For example, the microcavities 135 are arranged on the carrier substrate 115 in a honeycomb shape. Only optionally, the microfluidic device 100 comprises a buffer storage chamber 140 configured to store the lysis buffer and deliver it into the mixing chamber 110.

In other words, the approach presented here allows for fully automated and isolation-free quantification of circulating tumor cells from whole blood in a microfluidic environment. More specifically, a microfluidic counterpart to an isolation-free and hitherto only a manual process is provided, which comprises preparation of the blood sample until CTC quantification. The application is of particular interest for automated microfluidic systems such as the microfluidic device 100 described here, which offer analysis at a so-called point-of-care (PoC), i.e. subject in particular to time-critical boundary conditions and have only limited space available for reagents and sample material.

The process and microfluidic device 100 described in the Fig. below further generally permit detection of all nucleus-containing cells from whole blood, referred to here as sample liquid 105. This relates in particular to leukocytes, but also to endothelial cells and/or stem cells, so that alternative blood analyses can also be performed.

In accordance with this embodiment example, the core elements of the approach presented comprise a chronological composition of individual steps and/or components. For example, in an exemplary application, a selective lysis of the sample liquid 105 is performed using, for example, an ACK lysis buffer to maximize the density differences between lysed erythrocytes and non-lysed, still intact nucleus-containing cells. For example, to save additional time, the dyes required for subsequent detection and those still to be incubated are already added to the lysis buffer in parallel at this point, creating an “all-in-one buffer”. The nucleus information does not necessarily have to be provided via a bright-field transmitted light microscopy, in particular in the case that this possibility does not exist in the microfluidic device 100 or the associated optical path cannot be designed to be transparent enough. Instead, it is conceivable that the nucleus is fluorescently dyed for “nucleus present or not?” verification equivalently with a standard DNA dye. According to this embodiment example, the resulting and already partially dyed lysate, also referred to as blood lysate, is transferred into the relatively shallow and large area detection chamber 120, wherein the natural and homogeneously distributed sedimentation of all intact nucleus-containing cells over the detection area under lysed erythrocytes is awaited uniformly in a structure of microcavities 135 or micro-troughs located at the bottom of the chamber 120. This makes a negative selection “virtually isolation-free” and thus in the most ideal form possible. Note that the sedimentation time is used as the remaining incubation time for the dyes used, for example in the form of a parallelization.

CTC detection by fluorescence microscopy is possible, for example, after gentle flushing away of the erythrocyte film products, “EH” for short, and hemoglobin, “Hb” for short, which are formed by lysis and are for the most part not yet at the bottom of the chamber and act as an optical barrier, wherein the sedimented nuclear-containing cells remain in the microcavities 135 and/or are protected by them from being flushed away. On the other hand, if the light path is transparent enough, meaning the microfluidic system is transparent, and there is still no slope of the system, both the chip with microcavities 135 and the washing process is optional. In this case, the cells can sediment directly onto a planar and transparent carrier substrate 115. All information, including the nucleus information, can then be extracted by fluorescent dyes and optical detection on the substrate bottom, as the optical barrier is virtually non-existent in this case. If the overall optical barrier is thin and thus transparent enough due to the lysate, the described analysis is carried out without a chip with microcavities 135 and without a washing process via fluorescence channels, for example via reflected light microscopy. The presented approach reduces cell losses and/or cell damage, as CTC detection is (virtually) isolation-free, thus only small sample volumes are processed and CTC quantification can be standardized. Furthermore, thanks to microfluidic integrability, automation is possible, since the need for classical laboratory equipment that is not or hardly combinable with microfluidic environments, such as centrifuges, containers, vessels, etc., as well as manual processing steps with dead times between different process stations, is reduced. Furthermore optionally, a volume of necessary reagents and sample liquid 105 is reduced, as effective washing and subsequent quantification of dyed cells is also possible by microfluidic channels and minimized dimensions of the detection chamber 120 as well as by the carrier substrate 115 with microcavities 135 as their bottom, which can be designated as a chip, for example by a conventional reflected light microscope or optical detection system after reflected light setup with reduced working distance. Microcavities 135 offer the advantage of being able to operate the microfluidic device 100 against a slope.

FIG. 2 shows a flowchart of a process 200 according to an embodiment example for detecting nucleus-containing cells in a sample liquid of a patient. For example, the process 200 is controlled or performed in conjunction with a microfluidic device as it was described in FIG. 1. The nucleus-containing cells to be detected are, for example, tumor cells, leukocytes, endothelial cells or stem cells. The process 200 comprises a step 205 for providing, a step 210 for outputting, and a step 215 for identifying. In the step 205 of providing, a mixing signal is provided to an interface with a mixing device, wherein the mixing signal causes mixing of the sample liquid with a lysis buffer in the mixing chamber of the microfluidic device in order to obtain a lysate. For example, the sample liquid is mixed with the lysis buffer for a predetermined duration, which according to this embodiment example is at least five minutes. In the step 210 of outputting, an application signal is output that causes the lysate to be applied on the carrier substrate of the microfluidic device to obtain a cell sediment and a cell suspension of the lysate, for example after a minimum sedimentation time has elapsed. In the step 215 of identifying, the nucleus-containing cells are identified from the cell sediment, for example in order to detect dyed cells.

Only optionally, the process 200 comprises a step 220 of introducing the sample liquid into the microfluidic device, whereby, for example, the process 200 is initiated. For example, an amount of the sample liquid comprises 500 μl, of which 100 μl, for example, is mixed with the lysis buffer in the step 205 of providing. According to this embodiment example, the lysis buffer comprises a fluorescent dye for determining a cell type of the nucleus-containing cells. The fluorescent dye is optionally already contained in the lysis buffer or is supplied to the lysis buffer in an optional step 225 of supplying prior to the step 205 of providing. This ensures that the nucleus-containing cells receive the dye and thus become optically detectable.

According to this embodiment example, in the step 205 of providing, the mixing signal is provided that causes mixing of an amount of the lysis buffer depending on an amount of the sample liquid. This means that the amounts of the lysis buffer and the sample liquid ideally have a predetermined mixing ratio in order to obtain a meaningful result in the step 215 of identifying. According to this embodiment example, the process 200 comprises a step 230 of washing the lysate prior to the step 215 of identifying using a wash buffer to cause optical transparency of the lysate. The wash buffer is, for example, isotonic and/or PH neutral. Thus, it is easier to detect the nucleus-containing cells in the step 215 of identifying. According to this embodiment example, the nucleus-containing cells are optically detected and/or quantified from the cell sediment in the step 215 of identifying, for example using a microscope device.

In other words, according to this embodiment example, in the step 220 of introducing, the sample liquid also referred to as the blood sample is added to the microfluidic device. All subsequent steps continue to be carried out fully automated on-chip. In particular, microfluidic unit operations such as sample transport, mixing, washing, etc. occur according to a programmed sequence and without human intervention. In the step 205 of providing, within the relatively high mixing chamber, whole blood is mixed with the ACK lysis buffer in a suitable working ratio and continuously mixed during a necessary lysis time for a homogeneous concentration. The fluorescent dyes required for optical detection or classification of CTCs from leukocytes are already mixed with the lysis buffer in the respective working concentration. As this is a completed microfluidic system, the incubation of the dyes takes place in parallel or in the step 225 of supplying. Thus, no additional separate wait time is required for the incubation.

The process of hemolysis takes place until a hemoglobin concentration (Hb) outside the red blood cell is in equilibrium with the Hb concentration within the red blood cell. From this moment, the membrane becomes impermeable to a further flow of Hb as well as other comparatively large proteins. When hemolysis is complete, the resulting lysate is pumped into the shallow and large area detection chamber in the step 210 of outputting. The height of the chamber is selected such that the sedimentation time of the smallest nucleus-containing intact cells contained is limited to a predetermined maximum value, also referred to as PoC suitability. As erythrocytes have undergone a diffuse media exchange between the cell interior and exterior due to selective lysis, a net density difference between them and the surrounding medium is effectively composed only by the cell membrane and is thus virtually negligible. Subsequently, such lysed cells have a sedimentation rate of nearly zero and “float around in the medium”. Unlysed cells, in particular nucleus-containing cells that continue to have intact membrane function, on the other hand, retain a relatively large density difference to the surrounding medium and sediment at high rates compared to erythrocyte films. The carrier substrate structured with microcavities is optionally found at the bottom of the chamber. The effective detection area of the chip, as well as the dimensions and thus the total number of cavities on the carrier substrate within this effective area, are chosen such that separation of cells on the bottom in a monolayer is easily achievable once the intact nucleus-containing cells are sedimented into the cavities and form the cell sediment. Loading can be assisted by gently pumping the cell suspension back and forth, which also brings cells into the cavities that have previously been deposited on bars, for example. Ideally, all CTCs within the cavities are readily identified and differentiated from leukocytes in the step 215 of identifying. Furthermore, a scanning duration for a microscope, for example, is limited to a predetermined maximum value by the effective detection area of the chip.

In the optional step 230 of washing, a gentle washing process with wash buffer is further performed. The washing process is to be designed so that trapped cells are not swirled out of the microcavities. To this end, it must be ensured that a flow rate is selected small enough. Nevertheless, the washing process is fast enough to keep the overall process duration short. Furthermore, sufficient optical transparency is achieved in order to observe and reliably distinguish the fluorescently dyed cells within the microcavities, also referred to as troughs, from the background. This provides, for example, the most robust negative selection possible as well as a reduction of interfacial scattering due to the homogeneous distribution of Hb. Thus, an intensity weakening as the light passes through the optical barrier as a result of scattering effects is also drastically reduced and any traces of the lysate remaining due to an unideal washing procedure are acceptable.

FIG. 3 shows a flowchart of a process 200 according to an embodiment example for detecting nucleus-containing cells in a sample liquid of a patient. The process 200 depicted here corresponds to the process 200 described in FIG. 2. Only the process 200 according to this embodiment example is shown in a time course. This means that the step 205 of providing in accordance with this embodiment example is performed after the step 220 of introducing the sample liquid, also referred to as preparation. In accordance with this embodiment example, a duration of the process 200 is measured from the step 205 of providing. According to the flowchart shown here, the step 210 of outputting, which can also be referred to as “chip loading,” occurs after the predetermined period of time that according to this embodiment example is five minutes. Further optionally, 50 μl of blood lysate is pumped into the carrier substrate according to this embodiment example into the shallow detection chamber (which can effectively contain 10 μl of blood). According to this embodiment example, the step 230 of washing is performed after a further 10 minutes, referred to as minimum sedimentation time. According to this embodiment, the step 215 of identifying is performed after at least 20 minutes after the start of the time measurement.

In the following, the process 200 is described using exemplary dimensions and characteristics only:

For the most convenient handling and a microfluidic sample transport as reproducible as possible, an initially “large” blood volume of, for example >500 μl, is entered into the microfluidic system. For example, along with blood sampling, this is the only processing step that can be manually performed in the entire process 200. For example, immediately after sample input, 100 μl of the blood is transferred into the mixing chamber of the system with dimensions ˜ 13 mm× ˜ 13 mm× ˜ 10 mm for further processing. In this case, the volume of the ACK buffer necessary at least for selective lysis is 400 μl, for example. The “All-in-One Buffer” contains all the dyes necessary for fluorescent detection in the respective working concentrations for tumor cell specificity, propidium iodide (PI) for living-dead dyes and/or a dye advantageous for nucleus recognition. In order to counteract sedimentation and thus uneven cell concentrations, the cell suspension is continuously mixed until complete lysis, wherein a minimum lysis time is approximately 5 min. The necessary hydrodynamic pressure is to be designed such that no damage to the cells can be induced, for example by generating a reduced shear force.

Subsequently, for example, 50 μl of the dyed lysate is pumped into the shallow detection chamber with the same capacity. The volume is selected such that after sedimentation of all intact nucleus-containing cells, 10 μl of blood can be analyzed on the chamber bottom for final quantification. The dimensions of such an exemplary chamber are 12.5 mm×12.5 mm for an effective detection area of the carrier substrate so that the detection area is approximately 156 mm². For example, a chamber height is 320 μm. If the density and radius of the lightest and smallest sphere-like CTC to be expected are =1070 kg/m³ and =3 μm for calculating the smallest sedimentation rate, then according to the Stokes' sedimentation equation

$${u}_{sed} = \frac{2 ( {ρ}_{p} - {ρ}_{f} ) {r}^{2} g}{9 η}$$

in blood lysate with a density _{f}=1012 kg/m³ and viscosity η=1.68 mPa·s (data from measurements), maximum sedimentation times of approximately 7 min 52 s are to be expected within such a chamber. This allows the step 210 of outputting to be performed in <10 min. For example, if the diameter of the largest expected CTCs from solid tumors is 30 μm in an analysis, then cavities with 40 μm diameter and 28 μm depth are advantageous. For example, cavities are to be arranged in a classical matrix or also hexagonally densely packed as circles, hexagons or squares. In all configurations, the goal is to maximize the area of the cavities relative to the total area of the chip for the most efficient loading of the carrier substrate, i.e. to make the bars, also known as partitions, between adjacent cavities as small as possible, preferably <10 μm, such as 3 μm.

For example, a structured chip with microcavities comprises silicon that has been etched at the appropriate locations. However, to create cavities, for example, a photosensitive lacquer with the appropriate thickness can also be used, which, for example, has either been laminated onto a suitable substrate as a finished dry resist or has been flung up onto it as a lacquer and cured, and in a final processing step has been opened photolithographically by photomask and UV light at the desired locations. Alternatively, the cavities can also be formed by laser, such as by means of an UKP laser, from a polymer film that has been applied to a substrate in the appropriate thickness.

If a chip with the detection area 12.5 mm×12.5 mm is used with cavities arranged as hexagons and thus most densely packed in a honeycomb shape, and if the inner diameter of such cavities is, for example, 40 μm and the web width 3 μm, a carrier substrate with a total of approximately 97,600 cavities is obtained. 10 μl of blood contains between 40,000 and 110,000 leukocytes, which corresponds to a target blood equivalent for the final optical detection in the step 215 of identifying. Thus, with ideal loading, on average between 0.41 to 1.13 cells per trough can be expected, which corresponds to a relatively good separation. Ideally, one cell is found per trough.

The wash buffer used is thereby clear, which means optically transparent, isotonic and pH neutral in order to ensure biological compatibility with the cells. For example, a phosphate buffered saline (PBS) is suitable for this purpose, which can be stored upstream in reagent bars of the microfluidic device at low cost and with long-term stability without any problems.

If, for example, the 50 μl chamber volume is to be replaced 3 times within 5 minutes for the sake of safety to ensure sufficient optical transparency, i.e. if it is to be rinsed out with the wash buffer, this results in an average constant flow rate of 0.5 μl/s, for example. For a chamber with a face-in area and face-out area of 12.5 mm×320 μm, there is thus an average flow rate of 125 μm/s. Thus, according to this embodiment example, the tumor cell previously considered is not swirled out of a cavity with 40 μm width, 28 μm depth, and 3 μm wide bars after it has fully sedimented to the bottom. Thus, such a wash process is practical.

The quantification, that is to say the step 215 of identifying, is performed, for example, using a reflected light fluorescence microscope. Again, the cells relevant to the statistics here, as distinguished from leukocytes, are living tumor cells with nuclei. Overall, the step 215 of identifying is started, for example, after only around 20 min, which corresponds to a time saving by a factor of ˜ 3 compared to manual processing. Thereby (significantly) smaller reagent and sample volumes would be used.

Alternatively, prior to the sample input or here the output according to step 210, a selective erythrocyte lysis (RBC) is conceivable, which, when viewed hydrodynamically and biologically, converts RBCs into a form favorable for the detection process. The stiffness and effective cell diameter of RBCs are thereby significantly reduced and lysed RBCs can no longer maintain antigen expression, thereby avoiding clumping.

Furthermore, for example, the cell property used as a distinguishing criterion for the elimination of RBCs from leukocytes (WBC) and CTCs is cell density. Intact RBCs have an average density of approximately 1,110 kg/m³ and are thus somewhat heavier than nucleus-containing cells (WBCs and CTCs) with densities of between 1,070 kg/m³ and 1,090 kg/m³. It should be noted that in this state, both cell types, that is RBCs containing no nucleus as well as nucleus-containing WBCs and CTCs, have a significant density difference from the surrounding medium, which is water-like. The medium generally comprises ˜ 1000 kg/m³. Consequently, both cell types also sediment to the bottom at about the same rate and a specificity that can be used for spatial separation is not yet available. However, if the RBCs are lysed, they only have approximately 1 to 3% of the density of an intact erythrocyte, and thus, after diffuse media compensation between the cell interior and exterior during lysis in suspension, they assume a density difference of nearly zero. The actually still remaining density difference is thereby effectively only composed by the thin cell membrane. Thus, while nucleus-containing cells remain unaffected by lysis and maintain their density difference to the medium (sedimentation rate constant), RBCs have a sedimentation rate of nearly zero and float in suspension. WBCs and CTCs sediment through these floating RBC films to the bottom. After a sufficiently large sedimentation time and sedimentation height, this condition is now specific enough to be called at least approximately isolation-free, because even the smallest WBCs and CTCs can thus be laid out on a detection area without loss. According to this embodiment example, simple process standardization using minimum sample volumes and system dimensions is therefore possible.

FIG. 4 shows a schematic embodiment example of a cross-section of a detection chamber 120 of a microfluidic device. According to this embodiment example, a carrier substrate 115 and a lysate 400 are arranged in the detection chamber 120, for example as described in FIG. 1. Thereby the lysate 400 comprises a fluorescent dye for detecting and/or identifying nucleus-containing cells 402. This means, for example, that a type of cell of the nucleus-containing cells 402 can be detected from the selected dye, for example tumor cells 403, as they absorb the color contained in the dye better than other nucleus-containing cells 402, for example leukocytes 404.

The detection chamber 120 has a height 405 of, for example, 320 μm in which the lysate 400 is arranged. According to this embodiment example, a state is thus shown, which is achieved by the step of outputting, for example as described in one of FIG. 2 or 3. In accordance with this embodiment example, the carrier substrate 115 comprises the plurality of microcavities 135 that are evenly distributed on the carrier substrate 115 in accordance with this embodiment example. The microcavities 135 are thereby separated from adjacent microcavities 135 by partitions 410. The microcavities 135 are thereby formed as part of the carrier substrate 115. They have thereby a depth 415 of, for example, 28 μm and are configured to receive the nucleus-containing cells 402 as cell sediment after a sedimentation time has elapsed, as shown in the following Fig.

FIG. 5 shows a schematic embodiment example of a cross-section of a detection chamber 120 of a microfluidic device. For example, the carrier substrate 115 shown here corresponds to the carrier substrate 115 described in FIG. 4. Only differently, FIG. 5 shows a state within the detection chamber 120 that is reached according to this embodiment example in the step of outputting after the minimum sedimentation time. This means that according to this embodiment, the nucleus-containing cells 402 including tumor cells 403 sink due to their density difference in a cell suspension 500 and deposit as cell sediment 505 in the microcavities 135.

FIG. 6 shows an evaluation device 600 according to an embodiment example and a microfluidic device 100. The evaluation device 600 is configured to evaluate a cell sediment in the microfluidic device 100. For example, the microfluidic device 100 shown here corresponds to the microfluidic device 100 described in FIG. 1.

The evaluation device 600 comprises a mixing device 605 for mixing the sample liquid with the lysis buffer in the mixing chamber, as described for example in FIG. 1, to obtain a lysate. Furthermore, the evaluation device 600 comprises an identification unit 610 configured to identify the nucleus-containing cells from the cell sediment. Furthermore, the evaluation device 600 according to this embodiment example comprises a control unit 615 configured to control and/or perform the steps of a process for detecting nucleus-containing cells in a sample liquid of a patient as described in any of FIG. 2 or 3. The mixing device 605 is formed as a pumping device, for example, which is configured to mix the sample liquid with the lysis buffer. The identification unit 610 is formed as a microscope unit, for example, which comprises a light source. By this, the lysate is advantageously quantified after being placed on the carrier substrate 115. According to this embodiment example, the carrier substrate 115 is formed four-cornered, more specifically square, such that a width 620 and a length 625 of the carrier substrate 115 have equal dimensions. A depth 630 of the carrier substrate 115 is less than the length 625 and/or the width 620. According to this embodiment example, the carrier substrate 115 is shown magnified in an enlarged section 635 such that the plurality of microcavities 135 is illustrated.

According to this embodiment example, the control unit 615 is configured to provide a mixing signal 640 to an interface with the mixing device 605. The mixing signal 640 causes mixing of the sample fluid with the lysis buffer in the mixing chamber of the microfluidic device 100 in order to obtain the lysate. Furthermore, the control unit 615 is configured according to this embodiment example to output an application signal 645, for example to an interface with a device-external application unit 650, to cause the lysate to be applied onto the carrier substrate 115 of the microfluidic device 100, in order to obtain the cell sediment and a cell suspension of the lysate, for example, after a minimum sedimentation time has elapsed. Furthermore, the control unit 615 is configured to identify the nucleus-containing cells from the cell sediment, for example using an identification signal 655 and the identification unit 610.

FIG. 7 shows a schematic embodiment example of a cross-section of a detection chamber 120 of a microfluidic device. For example, the detection chamber 120 shown here corresponds to the detection chamber 120 described in one of FIG. 1, 4, or 5. According to this embodiment example, a snapshot during the optional step of washing is shown. This means that according to this embodiment example, a wash buffer 700 is introduced into the detection chamber 120, which has a flow 705 for washing the cell suspension 500. The microcavities 135 and their partitions 410 thereby prevent a rinsing away of the cell sediment 505.

FIG. 8 shows a schematic embodiment example of a cross-section of a detection chamber 120 of a microfluidic device. For example, the detection chamber 120 shown here corresponds to the detection chamber 120 described in FIG. 7. In accordance with this embodiment example, in other words, a snapshot of the step of identifying is depicted. According to this embodiment example, the wash phase of the lysate 400 shown in FIG. 7 is concluded. The light source 800 shown here represents the identification device described in FIG. 6, which is configured to detect and/or quantify the cell sediment 505. According to this embodiment example, a further magnified view 805 of the plurality of microcavities 135 is furthermore shown. The microcavities 135 are depicted from the top plan view. The microcavities 135 are furthermore rounded in accordance with this embodiment example and have a distance 810 from each other that is less than a diameter 815 of a microcavity 135. The distance 810 corresponds to a thickness of at least one of the partitions 410. The nucleus-containing cells 402 present in cell sediment 505 are only optionally dyed differently, whereby the type of cell is detectable.

FIG. 9a shows a schematic illustration of a flow path 900 of a liquid for a microfluidic device according to an embodiment example. The flow path 900 occurs in a microfluidic device, for example, which has a plurality of microcavities as was described in one of FIG. 1 or 4 to 8. According to this embodiment example, a cavity portion 905 and a chamber portion 910 of the detection chamber are shown. According to this embodiment example, the liquid flows linearly in the chamber portion 910 as it is swirled in the cavity portion 905. The cavity portion 905 thereby represents a flow behavior within a microcavity surrounded by partitions. This prevents the cell sediment from being entrained and/or damaged by the flow.

In other words, there is no risk of cell loss during rinsing to remove the blood lysate, which acts as an “optical barrier” to interfere with CTC detection when the cell is at the bottom of the cavity. There are greatly reduced flow rates within a cavity compared to the area above the chamber bottom. If a cell is thus located at the bottom of a cavity, it also experiences only greatly reduced Stokes' forces and is not rinsed away.

If an embodiment example comprises an “and/or” conjunction between a first feature and a second feature, this is to be read such that the embodiment example according to one embodiment comprises both the first feature and the second feature and according to a further embodiment comprises either only the first feature or only the second feature.

1. A process for detecting nucleus-containing cells in a sample liquid of a patient using a microfluidic device, comprising:
providing a mixing signal to an interface with a mixing device, wherein the mixing signal causes mixing of the sample liquid with a lysis buffer in a mixing chamber of the microfluidic device to obtain a lysate;
outputting an application signal which causes application of the lysate onto a carrier substrate of the microfluidic device to obtain a cell sediment and cell suspension of the lysate; and
identifying the nucleus-containing cells from the cell sediment.

2. The process according to claim 1, further comprising washing the lysate prior to the step of identifying using a wash buffer to cause an optical transparency of the lysate, wherein in the step of washing, the lysate is washed using the wash buffer which is isotonic and/or pH neutral.

3. The process according to claim 1, wherein in the step of providing, the mixing signal is provided which causes mixing of an amount of the lysis buffer dependent on an amount of the sample liquid.

4. The process according to claim 1, wherein in the step of identifying, the nucleus-containing cells are optically detected and/or quantified from the cell sediment.

5. The process according to claim 1, wherein:
in the step of providing, the mixing signal is provided to mix the sample liquid with the lysis buffer, and the lysis buffer comprises a fluorescent dye for determining a cell type of the nucleus-containing cells.

6. The process according to claim 1, further comprising introducing the sample liquid into the microfluidic device.

7. A process for detecting nucleus-containing cells in a sample liquid of a patient using a microfluidic device, comprising:
mixing the sample liquid with a lysis buffer in a mixing chamber of the microfluidic device to obtain a lysate;
applying the lysate onto a carrier substrate of the microfluidic device to obtain a cell sediment and cell suspension of the lysate; and
identifying the nucleus-containing cells from the cell sediment.

8. A control device configured to carry out and/or control the steps of a process according to claim 1 in corresponding units.

9. A microfluidic device for detecting nucleus-containing cells in a sample liquid of a patient, wherein the microfluidic device is formed as a lab-on-chip cartridge, and wherein the microfluidic device comprises:
a mixing chamber configured to receive the sample liquid and a lysis buffer in order to obtain a lysate;
a carrier substrate configured to apply the lysate, in order to obtain a cell sediment and cell suspension of the lysate; and
a detection chamber, wherein the carrier substrate is arranged and/or is configured to be arranged in the detection chamber.

10. A microfluidic device according to claim 9, wherein the detection chamber has a height that is less than a width and a length of the detection chamber.

11. A microfluidic device according to claim 9, wherein the carrier substrate comprises a plurality of microcavities.

12. A microfluidic device according to claim 9, wherein the microfluidic device comprises a buffer storage chamber configured to store the lysis buffer and output it into the mixing chamber.

13. An evaluation device for evaluating a cell sediment in a microfluidic device according to claim 9, wherein the evaluation device comprises:
a mixing device configured to mix the sample liquid with the lysis buffer in the mixing chamber in order to obtain a lysate; and
an identification unit configured to identify the nucleus-containing cells from the cell sediment.

14. A computer program configured to carry out and/or control the steps of the process according to claim 1.

15. A machine-readable storage medium on which the computer program according to claim 14 is stored.

16. The process according to claim 5, further comprising supplying the fluorescent dye in the lysis buffer prior to the step of providing.
